(19) United States
(12) Patent Application Publication
(10) Pub. No.: US 2026/0077173 A1
(43) Pub. Date: Mar. 19, 2026

(54) ANTIMICROBIAL CATHETER CAPS

(71) Applicant: Mozarc Medical US LLC, Minneapolis, MN (US)

(72) Inventors: Valentino J. Tramontano, Brockton, MA (US); Genevieve L. Gallagher, Mendota Heights, MN (US); Sean McDermott, Weymouth, MA (US); Stephen W. Murray, Attleboro, MA (US); David Thomashey, E. Walpole, MA (US); Kartik Sudhir Bondekar, Quincy, MA (US); Scott Jordan McDonald, Somerville, MA (US)

(21) Appl. No.: 19/331,485

(22) Filed: Sep. 17, 2025

Related U.S. Application Data
(60) Provisional application No. 63/696,634, filed on Sep. 19, 2024.

(51) Int. Cl. A61M 39/20; A61L 2/232; A61M 39/00

(52) U.S. Cl. CPC A61M 39/20; A61L 2/232; A61L 2103/15; A61M 2039/0027; A61M 2205/0205; A61M 2205/0238; A61M 2207/00

(57) ABSTRACT

A catheter cap includes a housing defining at least one cavity and an opening at a distal end of the housing, and an antimicrobial agent within the at least one cavity. The housing is configured to couple to a catheter luer at the distal end. The at least one cavity is configured to contain a catheter lock solution in contact with the catheter luer at the opening. The antimicrobial agent is present in an amount sufficient to maintain a minimum inhibitory concentration (MIC) of the antimicrobial agent in the catheter lock solution for a period of time between successive operations of the catheter luer.

### CROSS-REFERENCE TO RELATED APPLICATIONS

This application claims the benefit of U.S. Provisional Patent Application Ser. No. 63/696,634, filed Sep. 19, 2024, which is entitled, “ANTIMICROBIAL CATHETER CAPS” and is incorporated by reference herein in its entirety.

### TECHNICAL FIELD

The present technology is related to catheter caps.

### BACKGROUND

A medical catheter may be used to withdraw and introduce fluids to and from body cavities, ducts, blood vessels, and other hollow anatomical structures of a patient. As an example, a dialysis catheter may be used in hemodialysis procedures, in which blood is withdrawn from a blood vessel of a patient for treatment and subsequently returned to the blood vessel for circulation. The dialysis catheter may be frequently manipulated during dialysis sessions for connecting and disconnecting the dialysis machine, which may increase a likelihood of contamination. For example, bacteria can colonize the hub and, if aseptic techniques are not rigorously followed, be introduced directly into the bloodstream, leading to catheter-related bloodstream infections (CRBSIs). Infections can result in serious complications, including sepsis, prolonged hospital stays, and increased morbidity and mortality.

### SUMMARY

The present disclosure is directed to catheter caps, methods for fabricating catheter caps, and methods and systems for using catheter caps for disinfecting catheters. A catheter cap includes one or more cavities that contain an antimicrobial agent. When the catheter cap is connected to the catheter luer, a catheter lock solution that includes the antimicrobial agent inhibits bacterial growth at the catheter luer. The antimicrobial agent is loaded in the catheter cap in an amount sufficient to maintain a minimum inhibitory concentration (MIC) of the antimicrobial agent for a period of time between successive operations of the catheter luer. For example, the antimicrobial agent may include a coating that mixes with or releases into a catheter lock solution introduced into the cavity, or may be dissolved in a catheter lock solution that is already present in the cavity. In this way, antimicrobial caps described herein may inhibit microbial growth in a small form factor and in a state that maintains a relatively long shelf life.

In some examples, the disclosure describes a catheter cap that includes a housing defining at least one cavity and an opening at a distal end of the housing and an antimicrobial agent within the at least one cavity. The housing is configured to couple to a catheter luer at the distal end. The at least one cavity is configured to contain a catheter lock solution in contact with the catheter luer at the opening. The antimicrobial agent is present in an amount sufficient to maintain a minimum inhibitory concentration (MIC) of the antimicrobial agent in the catheter lock solution for a period of time between successive operations of the catheter luer.

In some examples, the disclosure describes a method for disinfecting a catheter luer using a catheter cap that includes attaching a distal end of a housing of the catheter cap to the catheter luer. The housing defines at least one cavity and an opening at a distal end of the housing. The at least one cavity contains a catheter lock solution in contact with the catheter luer at the opening. Prior to attaching the distal end of the housing, the at least one cavity includes an antimicrobial agent in an amount sufficient to maintain a minimum inhibitory concentration (MIC) of the antimicrobial agent in the catheter lock solution for a period of time between successive operations of the catheter luer.

In some examples, the disclosure describes a method for fabricating a catheter cap that includes positioning an antimicrobial agent in at least one cavity of a housing. The housing defines an opening at a distal end of the housing and is configured to couple to a catheter luer at the distal end. The at least one cavity is configured to contain a catheter lock solution in contact with the catheter luer at the opening. The antimicrobial agent is present in an amount sufficient to maintain a minimum inhibitory concentration (MIC) of the antimicrobial agent in the catheter lock solution for a period of time between successive operations of the catheter luer.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1A is a conceptual diagram of an example hemodialysis system that includes a hemodialysis catheter and a patient.

FIG. 1B is a cross-sectional conceptual diagram of the example hemodialysis catheter of FIG. 1A.

FIG. 1C is a conceptual block diagram of the example catheter cap of FIG. 1B.

FIG. 1D is a graph illustrating a concentration of an antimicrobial agent in a catheter lock solution over a period of time.

FIG. 2A is a perspective view diagram of an example antimicrobial cap that includes an antimicrobial agent coated on a surface.

FIG. 2B is a cross-sectional side view diagram of the example antimicrobial cap of FIG. 2A.

FIG. 2C is a perspective view diagram of an example antimicrobial cap that includes an antimicrobial agent coated on a surface of a back cavity.

FIG. 2D is a cross-sectional side view diagram of the example antimicrobial cap of FIG. 2C.

FIG. 3A is a perspective view diagram of an example antimicrobial cap that includes an antimicrobial agent coated on a surface of an insert in a proximal cavity.

FIG. 3B is a cross-sectional side view diagram of the example antimicrobial cap of FIG. 3A.

FIG. 3C is a perspective exploded view diagram of an example antimicrobial cap that includes a plurality of fins as an insert in a proximal cavity.

FIG. 3D is a perspective exploded view diagram of an example antimicrobial cap that includes a plurality of tubes as an insert in a proximal cavity.

FIG. 3E is a perspective exploded view diagram of an example antimicrobial cap that includes a film as an insert in a proximal cavity.

FIG. 3F is a perspective exploded view diagram of an example antimicrobial cap that includes a foam as an insert in a proximal cavity.

FIG. 4A is a perspective view diagram of an example antimicrobial cap that includes a connector assembly and an antimicrobial agent coated on a surface of an insert in a proximal cavity.

FIG. 4B is a cross-sectional side view diagram of the example antimicrobial cap of FIG. 4A.

FIG. 4C is a perspective exploded view diagram of an example antimicrobial cap that includes a plurality of fins as an insert in a proximal cavity.

FIG. 4D is a perspective exploded view diagram of an example antimicrobial cap that includes a film as an insert in a proximal cavity.

FIG. 4E is a perspective exploded view diagram of an example antimicrobial cap that includes a foam as an insert in a proximal cavity.

FIG. 4F is a perspective view diagram of an example antimicrobial cap that includes a connector assembly and an antimicrobial agent coated on a surface of a tubular proximal cavity.

FIG. 4G is a cross-sectional side view diagram of the example antimicrobial cap of FIG. 4F.

FIG. 5A is a perspective view diagram of an example antimicrobial cap that includes an antimicrobial agent in a proximal cavity in an uncompressed state.

FIG. 5B is a cross-sectional side view diagram of the example antimicrobial cap of FIG. 5A.

FIG. 5C is a perspective view diagram of an example antimicrobial cap that includes an antimicrobial agent in a proximal cavity in a compressed state.

FIG. 5D is a cross-sectional side view diagram of the example antimicrobial cap of FIG. 5C.

FIG. 6A is a flow diagram illustrating an example technique for disinfecting a catheter luer using an antimicrobial cap that includes receiving a catheter lock solution.

FIG. 6B is a flow diagram illustrating an example technique for disinfecting a catheter luer using an antimicrobial cap that includes injecting a catheter lock solution.

FIG. 6C is a flow diagram illustrating an example technique for disinfecting a catheter luer using an antimicrobial cap that includes releasing a catheter lock solution.

### DETAILED DESCRIPTION

The present disclosure is directed to catheter caps, methods for fabricating catheter caps, and methods and systems for using catheter caps for disinfecting catheters.

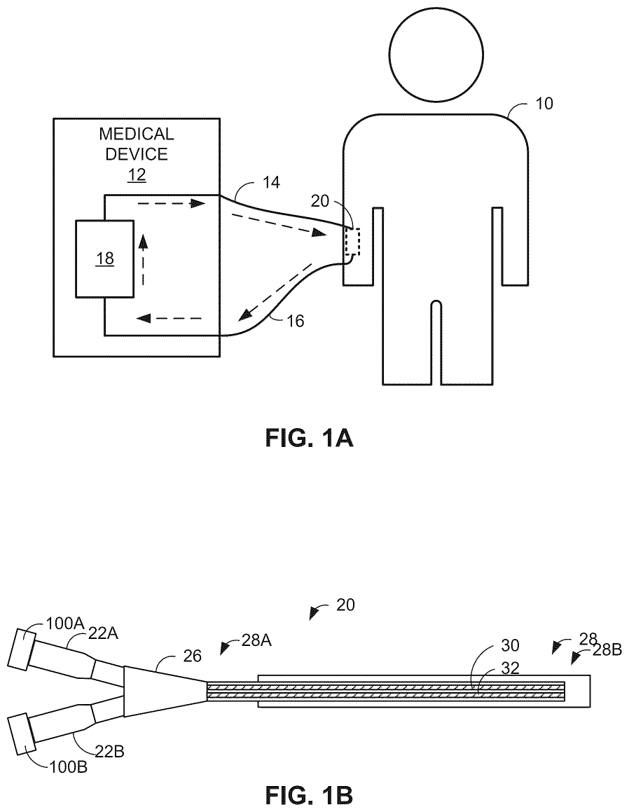

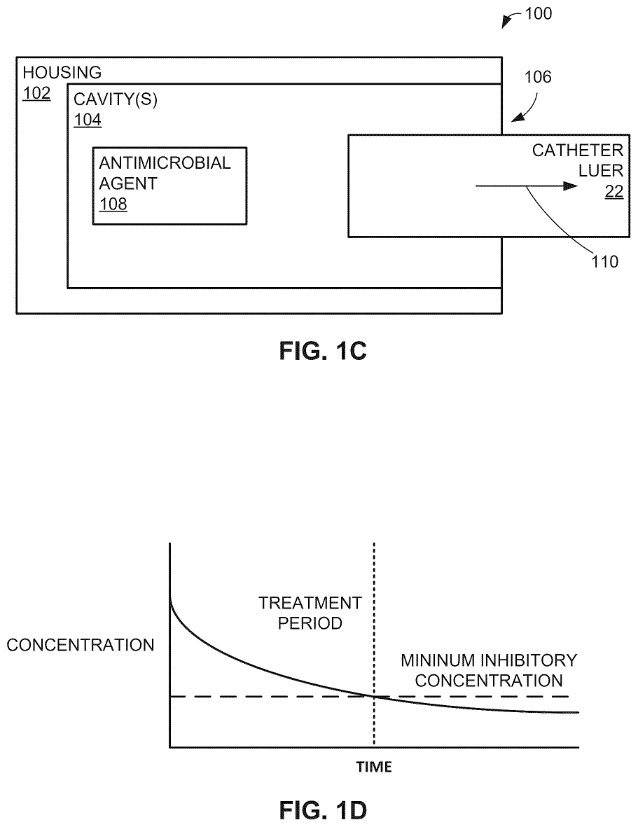

Intravascular devices used for chronic vascular access may be kept within vasculature of a patient for extended periods of time, such that interfaces of the intervascular devices may be exposed to bacteria and other organisms, and for which antimicrobial caps as described herein may reduce proliferation of such organisms. FIG. 1A is a conceptual diagram of an example hemodialysis system and patient 10. However, the antimicrobial caps described herein may be used with other systems, e.g., in which an intravascular device remains in contact with blood for an extended period of time.

A clinician (or other user) may fluidically connect an aspiration line 16 to an inflow port (not shown) on a medical device 12 and to an intravascular device 20 in patient 10 to provide access to the vasculature of patient 10. Aspiration line 16 may be configured to facilitate the transport of blood from intravascular device 20 to medical device 12. For example, blood from patient 10 may contain high levels of waste products due to kidney failure or kidney disease. The clinician may also fluidically connect a perfusion line 14 to an outflow port (not shown) of the medical device 12 and to intravascular device 20 in patient 10 to provide access to a vein of patient 10. Perfusion line 14 may be configured to return relatively cleaner blood from medical device 12 to intravascular device 20.

Medical device 12 is configured to remove waste products from the blood received via aspiration line 16. For example, medical device 12 may include a dialyzer 18 and/or one or more filters that may remove waste products and excess fluid from the blood received via aspiration line 16. Dialyzer 18 may use a dialysate solution to remove the waste products and excess fluid from the blood of patient 10. Medical device 12 may also include a blood pump (not shown) which is configured to keep the blood of patient 10 flowing through medical device 12.

Intravascular device 20 may be a vascular access device, such as an intravenous catheter (e.g., to a lumen of the catheter), an arteriovenous fistula, or a synthetic graft (not shown). The arteriovenous fistula or the synthetic graft in the patient may be accessed, for example, via a needle or cannula. FIG. 1B is a cross-sectional conceptual diagram of an example hemodialysis catheter assembly as intravascular device 20 of FIG. 1A that includes antimicrobial catheter caps 100A, 100B. Intravascular device 20 includes an elongated body 28 and a proximal hub 26 that includes two luers—an aspiration luer 22A and a perfusion luer 22B. Aspiration luer 22A is configured to fluidically couple to aspiration line 16, while perfusion luer 22B is configured to fluidically couple to perfusion line 14. Elongated body 28 defines a proximal portion 28A, a distal portion 28B, and one or more lumens; in the example of FIG. 1B, elongated body 28 includes an aspiration lumen 30 fluidically coupled to the aspiration luer 22A and a perfusion lumen 32 fluidically coupled to perfusion luer 22B. Each luer 22A, 22B is covered by a catheter cap 100A, 100B, respectively. Intravascular device 20 may be placed in patient 10 for an extended period of time, such as on the order of weeks or months. Between successive operations of luers 22, each luer 22 may be filled with a catheter lock solution and covered by a respective cap 100. If left untreated, bacteria may grow within the catheter lock solution within luers 22.

According to examples described herein, cap 100 may be configured to inhibit growth of microbes, such as bacteria and/or fungi, in and/or on luers 22. FIG. 1C is a conceptual block diagram of an example catheter cap 100 of FIG. 1B. Cap 100 includes a housing 102 defining at least one cavity 104 and an opening 106 at a distal end (i.e., toward luer 22) of housing 102. Housing 102 may be formed from any of a variety of materials, such as polypropylene, acrylonitrile butadiene styrene (ABS), or another material that is suitable for contact with a fluid that may contact a patient. Housing 102 is configured to couple to a catheter luer 22 at the distal end of housing 102. Housing 102 may include standard luer connection types, such as a male luer attachment compliant to ISO 80369-7 or other connection type configured to interface with a catheter luer.

Catheter luer 22 may be positioned in a patient for a relatively long period of time, including outside of a clinical setting. To enable more comfortable use of catheter luer 22 and reduce a likelihood of catheter luer 22 becoming snagged against an object, cap 100 may have a relatively small form factor having a small volume. For example, a volume of cavity(s) 104 may be less than one milliliter.

Cavity(s) 104 is configured to contain a catheter lock solution 110 in contact with catheter luer 22 at opening 106. As will be described further below, lock solution 110 may be received from luer 22 or another injection device, or may be contained in cavity 104 along with antimicrobial agent 108. Lock solution 110 may include a variety of sterile, water-based solvents, such as heparinized saline or citrate buffer.

To inhibit microbial growth in lock solution 110, cap 100 includes an antimicrobial agent 108 within cavity(s) 104. Antimicrobial agent 108 may be configured to inhibit growth of microbes, such as bacteria and fungi. For catheter caps 100 used for hemodialysis catheters, antimicrobial agent 108 may be configured to inhibit growth of inhibit microbes commonly known to cause infections of hemodialysis catheters, such as staphylococcus aureus and/or klebsiella pneumoniae. A variety of antimicrobial agents may be used for antimicrobial agent 108 including, but not limited to, rifampin, chlorhexidine acetate, minocycline, cefazolin, gentamicin, silver glass, or silver salt.

Antimicrobial agent 108 is present in cavity(s) 104 in an amount sufficient to maintain a minimum inhibitory concentration (MIC) of antimicrobial agent 108 in catheter lock solution 110 for a period of time between successive operations of catheter luer 22. FIG. 1D is a graph illustrating a concentration of an antimicrobial agent in a catheter lock solution over a period of time. While the example of FIG. 1D illustrates a generally exponential decrease in a concentration of antimicrobial agent 108, such concentration may depend on a mechanism of release of antimicrobial agent 108 (e.g., a powder coating vs. a controlled-release coating), such as will be explained further below.

As illustrated in FIG. 1D, antimicrobial agent 108 may be contained or released into lock solution 110 at an initial concentration. Over time, a concentration of antimicrobial agent 108 in lock solution 110 may decrease. As one example, antimicrobial agent 108 may be consumed or inactivated by microbials, reducing the concentration of antimicrobial agent 108. As another example, antimicrobial agent 108 may undergo hydrolysis, oxidation, photodegradation, pH degradation, temperature degradation, or other reaction or degradation mechanism that causes breakdown of antimicrobial agent 108. As such, antimicrobial agent 108 may be present in an amount or at a release rate sufficient to maintain a concentration above a minimum inhibitory concentration for a period of time corresponding to a treatment period. This treatment period may be a period between successive operations of catheter luer 22. For example, some patients with access to in-home dialysis may undergo dialysis treatment daily, such as at night. In some examples, the treatment period may be at least one day. In some examples, the treatment period may be at least three days. For example, some patients accessing dialysis at a clinic may go to the clinic two or more times each week.

In such examples, antimicrobial agent 108 may be present in an amount sufficient to maintain an MIC of 16 micrograms per milliliter (μg/mL) in lock solution 110 for the period of time, which may generally be sufficient to inhibit growth of bacteria commonly encountered at catheter luer 22. However, an MIC may vary depending on a composition of antimicrobial agent 108 and a solubility of antimicrobial agent 108 in lock solution 110. In some examples, antimicrobial agent 108 is present in an amount sufficient to maintain an MIC of 1 μg/mL for rifampin, 20 milligrams per milliliter (mg/mL) for chlorhexidine, or 50 mg/ml for one of minocycline, cefalin, or gentamicin.

In some examples, antimicrobial agent 108 includes a powder coating. For example, a powder may be configured to remain on surfaces of cavity(s) 104 up to receiving lock solution 110, disperse relatively easily in the presence of lock solution 110, and dissolve into lock solution 110. As a result, a concentration of antimicrobial agent 108 in lock solution 110 may generally correspond to an overall loading by weight of antimicrobial agent 108 in cavity(s) 104 and a volume of lock solution 110 contained by cavity(s) 104.

In some examples, the powder is a precipitated (or solvent-based) powder coating that forms through precipitation by the evaporation of a solvent. For example, antimicrobial agent 108 may be dissolved into a solvent, such as alcohol, to form a solution. The solution may be injected into cavity(s) 104. The solvent may be evaporated, and as the solvent evaporates, antimicrobial agent 108 precipitates out of the solution and forms a solid layer on the surface of cavity(s) 104.

In some examples, antimicrobial agent 108 is incorporated into a polymeric coating configured to release antimicrobial agent 108 over a period of time. The polymeric coating may include antimicrobial agent 108 encapsulated in a water permeable polymer matrix. Encapsulating antimicrobial agent 108 in the polymer matrix may help to control the release of the antimicrobial agent over a long term to maintain antimicrobial efficacy of antimicrobial agent 108. Antimicrobial agent 108 may be present as particles in the polymer matrix. Molecules of antimicrobial agent 108 may be released from the particles and may diffuse or otherwise migrate from an interior of the coating to a surface and leach into the lock solution 110.

The polymer matrix may be configured to release antimicrobial agent 108 into lock solution 110 according to a desired release (or elution) rate. This release rate may depend on a variety of factors including, but not limited to, a composition and water permeability of the polymer matrix, a composition and solubility of antimicrobial agent 108, a size of the particles of antimicrobial agent 108, and the like. For example, more soluble antimicrobial agents such as chlorhexidine gluconate will have a high release rate whereas the relatively water insoluble hydrochloride salt releases slowly. A variety of polymers may be used for the polymer matrix including, but not limited to, a polyurethane, such as a thermoplastic polyurethane elastomer, a polyester, polylactic acid, polyglycolic acid, polytetramethylene glycol, polyacrylamide, polyacrylic acid, polyacrylate, poly(2-hydroxyethyl methacrylate), polyethylene-imine, poly-sulfonate and copolymers thereof such as poly(lactic acid-co-glycolic acid) (PLA/PGA), polyacrylic-co-hydroxylated-acrylate, poly(acrylic acid-co-2-hydroxy ethyl methacrylate). A variety of antimicrobial agents may be used for the particles of antimicrobial agent 108 including, but not limited to, silver-based antimicrobial agents; polybiguanides and salts thereof; chlorhexidine and salts thereof such as the dihydrochloride, diacetate and digluconate salt of chlorhexidine; hexachlorophene; cyclohexidine; chloroaromatic compounds such as triclosan; para-chloro-meta-xylenol. Suitable antimicrobial formulations may include one or more of those antimicrobial formulations described in PCT Patent Application No. PCT/US2018/062218, filed Nov. 21, 2018, and U.S. patent application Ser. No. 14/567,183, filed Dec. 11, 2014, the entire content of each of these applications is incorporated herein by reference.

In some examples, antimicrobial agent 108 is dissolved in catheter lock solution 110. For example, antimicrobial agent 108 may be dissolved at a concentration that is sufficient to maintain the MIC for the desired period of time. As such, catheter lock solution 110 may not be provided from an external source, thereby reducing a likelihood of contamination.

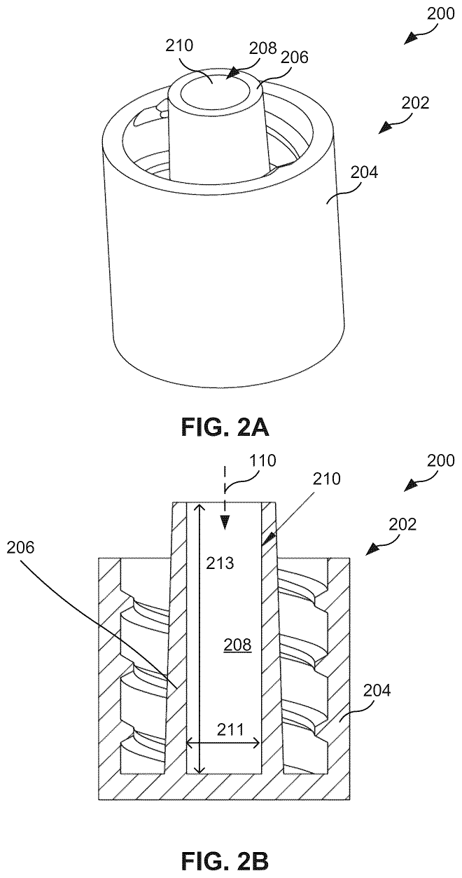

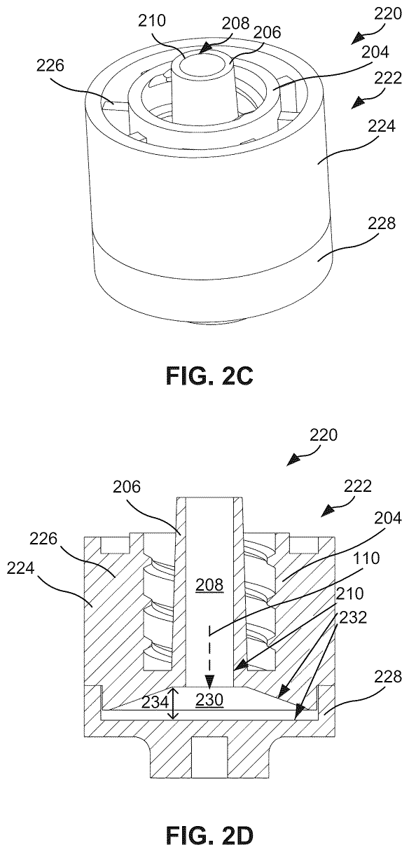

As described above, antimicrobial agents may be present as precipitated or controlled-release coatings, or within the catheter lock solution. As such, a loading of the antimicrobial agents may be related to an amount of surface area or volume available to deposit a corresponding coating and/or provide a corresponding solution of the antimicrobial agents. As will be described further in the examples of FIG. 2-5, catheter caps may be configured to increase an amount of surface area or volume available for antimicrobial agents, and/or provide various ways of providing or receiving a catheter lock solution.

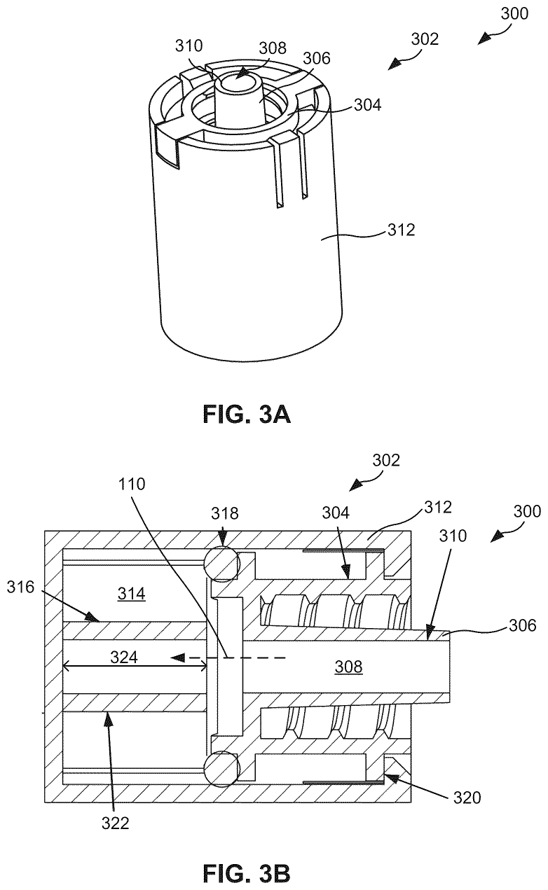

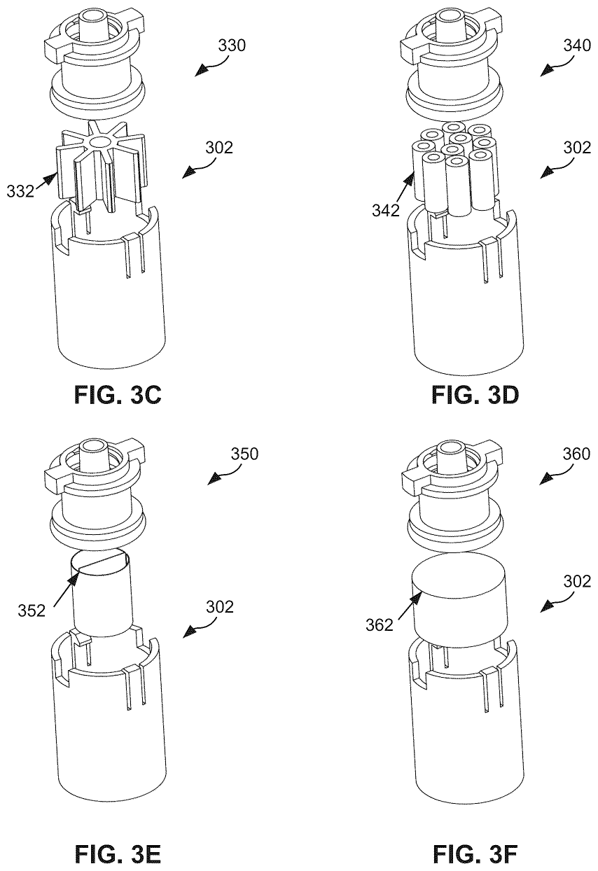

In some examples, such as will be described with respect to FIG. 2A-2D and 3A-3F, a catheter lock solution may be provided by a catheter luer, and an antimicrobial agent may be present as a coating on various surfaces on or within the catheter cap. By receiving a catheter lock solution from the catheter luer, the antimicrobial agent may be provided in a concentrated form, and the volume of the catheter cap reduced.

FIG. 2A is a perspective view diagram of an example antimicrobial catheter cap 200 that includes an antimicrobial agent coated on a surface of a cavity, while FIG. 2B is a cross-sectional side view diagram of the example antimicrobial catheter cap 200 of FIG. 2A. Catheter cap 200 includes a housing 202. Housing 202 includes a connector housing 204 and a hollow projection 206 consistent with a connection luer type; however, other connection luer types may be used as a connection assembly for interfacing with a catheter luer. Connector housing 204 includes an opening at a distal end of housing 202 and is configured to interface with a catheter luer, such a catheter luer 22 of FIG. 1C. For example, an inner surface of connector housing 204 includes threads configured to interface with corresponding threads of a catheter luer. Hollow projection 206 may be positioned in connector housing 204, such as in a radial middle of connector housing 204. Hollow projection 206 includes an inner cavity 208 defined by a radially inner surface 210. Inner cavity 208 may have a diameter 211 and length 213 that together define a surface area of inner cavity 208.

Inner cavity 208 is configured to receive catheter lock solution 110 from a catheter luer. An antimicrobial agent is coated on radially inner surface 210 of hollow projection 206 and configured to release into catheter lock solution 110 in inner cavity 208. In operation, catheter lock solution 110 may flow into inner cavity 208 and cause at least a portion of the antimicrobial agent to migrate into catheter lock solution 110 from radially inner surface 210, resulting in a concentration of the antimicrobial agent in catheter lock solution 110. As explained above, the antimicrobial agent is present on radially inner surface 210 in an amount sufficient to maintain a minimum inhibitory concentration (MIC) of the antimicrobial agent in catheter lock solution 110 for a period of time between successive operations of the catheter luer. Radially inner surface 210 may have a high surface area for accommodating the antimicrobial agent in this sufficient amount. For example, diameter 211 and/or length 213 may be selected to produce a desired surface area for the antimicrobial agent. As a result, a relatively high amount of antimicrobial agent may be contained on radially inner surface 210 in a relatively small volume, such as an additional cavity behind inner cavity 208.

In some examples, an antimicrobial agent may be present as a coating on additional and/or alternative surfaces of a catheter cap. FIG. 2C is a perspective view diagram of an example antimicrobial cap 220 that includes a back cavity 230, while FIG. 2D is a cross-sectional side view diagram of the example antimicrobial cap of FIG. 2C. Catheter cap 220 includes a housing 222. Unless otherwise indicated, housing 222 includes various features similar to housing 202 of FIGS. 2A and 2B, such as connector housing 204, hollow projection 206, inner cavity 208, and inner radial surface 210.

Housing 222 also includes an outer housing 224 attached to connector housing 204 of housing 222 by plurality of fins 226 and a back housing 228 attached to outer housing 224. Outer housing 224, plurality of fins 226, and/or back housing 228 may be configured to provide additional volume for creating a back cavity 230 proximal to inner cavity 208. Back cavity 230 and/or hollow projection 206 may define one or more coating surfaces for depositing the antimicrobial agent. For example, the antimicrobial agent may be coated on at least one of a radially inner surface 210 of hollow projection 206 or surfaces 232 of back cavity 230. Back cavity 230 may be configured to receive catheter lock solution 110 from inner cavity 208 of hollow projection 206. Dimensions of back cavity 230, such as a depth 234, may be selected to increase a surface area of back cavity 230. In some examples, hollow projection 206 may be configured with a higher surface area. For example, hollow projection 206 may extends distally past the opening at the distal end of housing 222, thereby increasing a surface area available to deposit the antimicrobial coating.

In operation, catheter lock solution 110 may flow into inner cavity 208 and cause at least a portion of the antimicrobial agent to migrate into catheter lock solution 110 from contact surfaces, such as radially inner surface 210 of hollow projection 206 and/or surfaces 232 of back cavity 230. As explained above, the antimicrobial agent is present on surfaces 210 or 232 in amounts sufficient to maintain a minimum inhibitory concentration (MIC) of the antimicrobial agent in catheter lock solution 110 for a period of time between successive operations of the catheter luer.

In some examples, catheter caps described herein may include further structures configured to increase a surface area available for a coating of an antimicrobial agent. FIG. 3A is a perspective view diagram of an example antimicrobial cap that includes an antimicrobial agent coated on a surface of an insert in a proximal cavity, while FIG. 3B is a cross-sectional side view diagram of the example antimicrobial cap of FIG. 3A. Cap 300 includes a housing 302. Unless otherwise indicated, housing 302 includes a connector housing 304, a hollow projection 306, an inner cavity 308, and a radially inner surface 310 similar to connector housing 204, hollow projection 206, inner cavity 208, and radially inner surface 210 of FIGS. 2A and 2B.

Housing 302 further includes cap body 312 defining a proximal cavity 314 at a proximal end of housing 302. Proximal cavity 314 is in in fluid communication with the opening via inner cavity 308. An O-ring 318 may be positioned against connector housing 304 along an inner surface of cap body 312 to seal proximal cavity 314, while a snap member 320 may secure connector housing 204 within cap body 312.

The antimicrobial agent is coated on one or more surfaces within proximal cavity 314. In the example of FIG. 3B, cap 300 further includes an insert 316 positioned within proximal cavity 314. The antimicrobial agent is coated on a surface 322 of insert 316. In operation, catheter lock solution 110 may flow into proximal cavity 314 via inner cavity 308 and cause at least a portion of the antimicrobial agent on surface 322 of insert 316 to migrate into catheter lock solution 110, resulting in a concentration of the antimicrobial agent. The antimicrobial agent is present on surface 322 in an amount sufficient to maintain a minimum inhibitory concentration (MIC) of the antimicrobial agent in catheter lock solution 110 for a period of time between successive operations of the catheter luer. Surface 322 may have a high surface area for accommodating the antimicrobial agent in this sufficient amount. For example, depth 324 of insert 316 may be selected to produce a desired surface area for the antimicrobial agent. As a result, a relatively high amount of antimicrobial agent may be contained on surface 322 in a relatively small volume.

Additionally, a surface area of insert 316 may be related to a form of insert 316. FIG. 3C is a perspective exploded view diagram of an example antimicrobial cap 330 that includes a plurality of fins 332 as an insert in a proximal cavity. Cap 330 includes housing 302 and plurality of fins 332 positioned within housing 302. For example, plurality of fins may be extruded from polyurethane or another biocompatible polymer into a desired shape and cut to size according to proximal cavity 314. A surface area of plurality of fins 332 may be high due to a number of fins in contact with a catheter lock solution. While plurality of fins 332 includes eight fins, any number of fins may be used.

FIG. 3D is a perspective exploded view diagram of an example antimicrobial cap 340 that includes a plurality of tubes 342 as an insert in a proximal cavity. Cap 340 includes housing 302 and plurality of tubes 342 positioned within housing 302. For example, plurality of tubes 342 may include polyurethane or another biocompatible polymer capable of being coated with antimicrobial agent. A surface area of plurality of tubes 342 may be high due to an inner and outer surface of plurality of tubes 342 and a number of tubes 342 in contact with a catheter lock solution. While plurality of tubes 342 includes ten tubes, any number of tubes may be used.

FIG. 3E is a perspective exploded view diagram of an example antimicrobial cap 350 that includes a film 352 as an insert in a proximal cavity. Cap 350 includes housing 302 and film 352 positioned within housing 302. Film 352 may include a cast film formed from the time-release polymer matrix described in FIG. 1C above, and may include a variety of shapes.

FIG. 3F is a perspective exploded view diagram of an example antimicrobial cap 360 that includes a foam 362 as an insert in a proximal cavity. Cap 360 includes housing 302 and foam 362 positioned within housing 302. Foam 362 includes an antimicrobial agent in a polymer matrix, such as encapsulated particles in the polymer matrix or a coating on the polymer matrix, that defines a plurality of void volumes into which the catheter lock solution may flow. The void volumes may create a relatively high surface area for coating or encapsulating the antimicrobial agent. Suitable foams may include one or more of those foams described in U.S. patent application Ser. No. 17/516,262, entitled “ANTIMICROBIAL FOAM ARTICLES” and filed Nov. 21, 2021, the entire content of which is incorporated herein by reference.

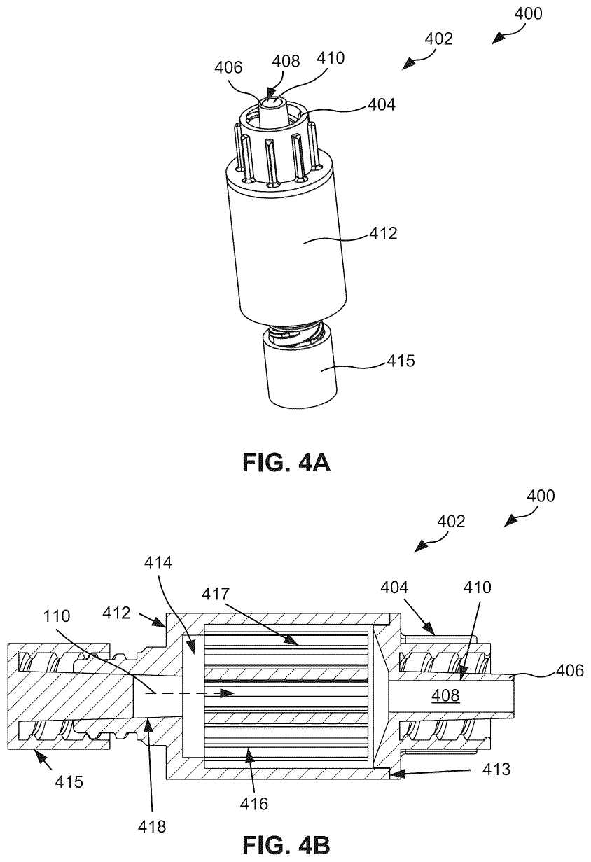

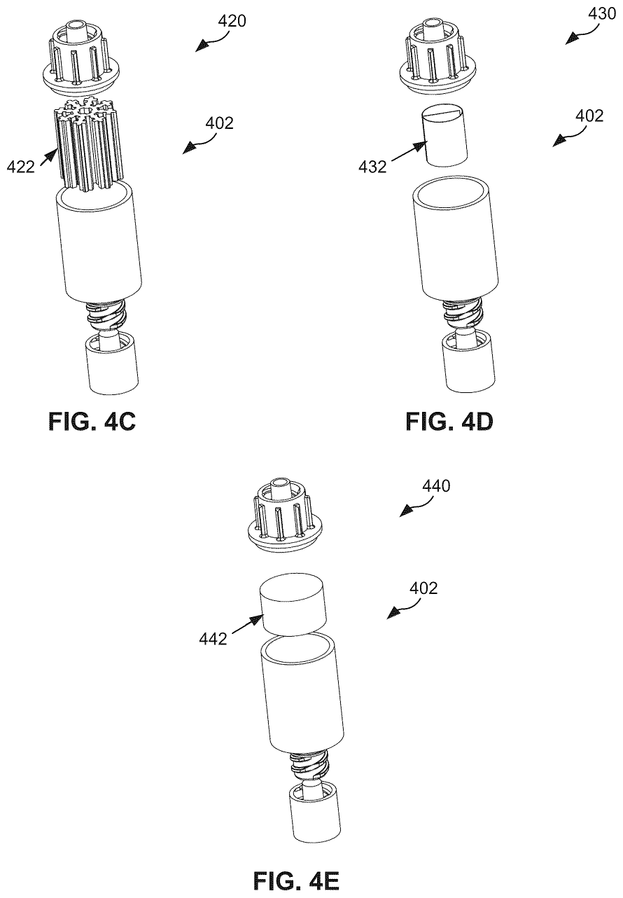

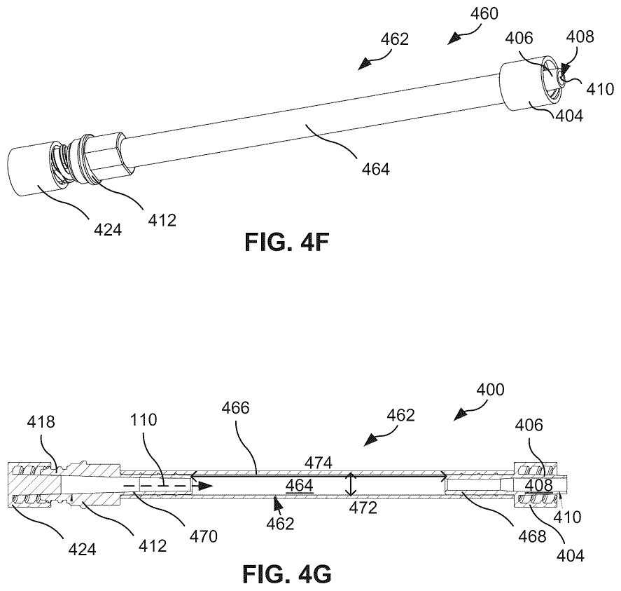

In the examples of FIG. 2A-2D and 3A-3F, catheter caps received a catheter lock solution from the catheter luer. However, in the examples of FIG. 4A-4G described below, another device may supply the catheter lock solution, such as an injection device capable of in-line mixing of the antimicrobial agent into the catheter lock solution. Such in-line mixing may improve incorporation and mixing of the antimicrobial agent into the catheter lock solution.

FIG. 4A is a perspective view diagram of an example antimicrobial cap that includes a connector assembly and an antimicrobial agent coated on a surface of an insert in a proximal cavity, while FIG. 4B is a cross-sectional side view diagram of the example antimicrobial cap of FIG. 4A. Cap 400 includes a housing 402. Unless otherwise indicated, housing 402 includes a distal connector housing 404, a hollow projection 406, an inner cavity 408, and a radially inner surface 410 similar to connector housing 204, hollow projection 206, inner cavity 208, and radially inner surface 210 of FIGS. 2A and 2B.

Housing 402 also includes a proximal connector housing 412 at a proximal end of housing 402. Proximal connector housing 412 defines a proximal cavity 414 and is coupled to distal connector housing 404, such as through adhesive 413. Proximal cavity 414 is in fluid communication with the opening via inner cavity 408. Proximal connector housing 412 includes a proximal projection 418 that is configured to interface with an injection device and receive catheter lock solution 110 from the injection device into proximal cavity 414 of housing 402. Housing 402 also includes a temporary catheter cap 415.

In the example of FIGS. 4A and 4B, cap 400 also includes an insert 416 positioned within proximal cavity 414. Antimicrobial agent is coated on surfaces of insert 416 and configured to release into catheter lock solution 110 in proximal cavity 414. In operation, catheter lock solution 110 may flow into proximal cavity 414 and cause at least a portion of the antimicrobial agent to migrate into catheter lock solution 110 from the surface of insert 416. Once lock solution is in proximal cavity 414, cap 424 may seal catheter lock solution 110.

Insert 416 may be similar to insert 316 of FIGS. 3A and 3B. FIG. 4C is a perspective exploded view diagram of an example antimicrobial cap 420 that includes a plurality of fins 422 in a housing 402 as an insert in a proximal cavity, FIG. 4D is a perspective exploded view diagram of an example antimicrobial cap 430 that includes a film 432 in a housing 402 as an insert in a proximal cavity, and FIG. 4E is a perspective exploded view diagram of an example antimicrobial cap 440 that includes a foam 442 in a housing 402 as an insert in a proximal cavity. Plurality of fins 422, film 432, and foam 442 may be similar to plurality of fins 332, film 352, and foam 362 of FIGS. 3C, 3E, and 3F, respectively.

FIG. 4F is a perspective view diagram of an example antimicrobial cap 460 that includes an antimicrobial agent coated on a surface of a tubular proximal cavity 464, while FIG. 4G is a cross-sectional side view diagram of the example antimicrobial cap of FIG. 4F. Cap 460 includes a housing 462. Unless otherwise specified, distal connector housing 404, hollow projection 406, inner cavity 408, radially inner surface 410, proximal connector housing 412, and proximal projection 418 may be similar to similarly named components of housing 402 of FIGS. 4A and 4B.

Cap 460 further includes a tubular housing 462 coupled to an extension 468 of distal connector housing 404 and an extension 470 of proximal connector housing 412. Tubular housing 462 defines a tubular proximal cavity 464 that is configured to receive catheter lock solution 110 from a catheter luer. Proximal cavity 464 may have a diameter 472 and length 474 that together define a surface area of proximal cavity 464. Antimicrobial agent is coated on an inner surface 466 of tubular proximal cavity 464 and configured to release into catheter lock solution 110 in proximal cavity 464. In operation, catheter lock solution 110 may flow into proximal cavity 464 and cause at least a portion of the antimicrobial agent to migrate into catheter lock solution 110 from inner surface 466. Once lock solution is in proximal cavity 464, cap 424 may seal catheter lock solution 110.

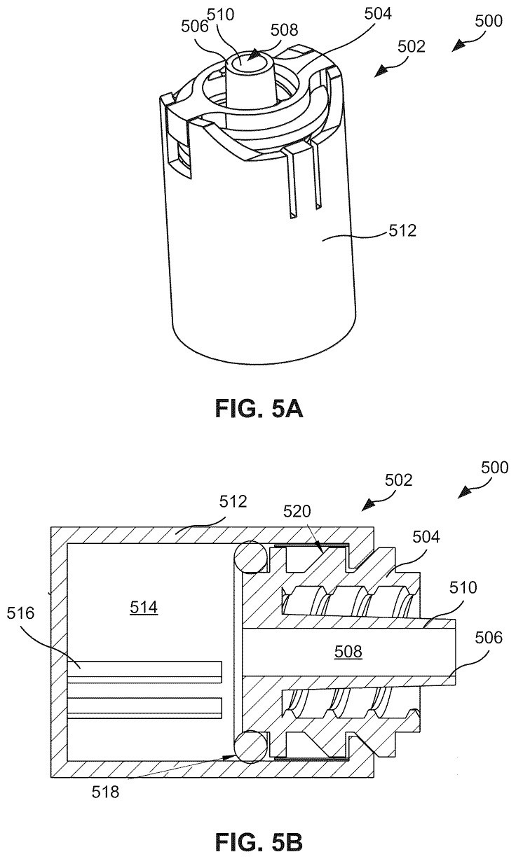

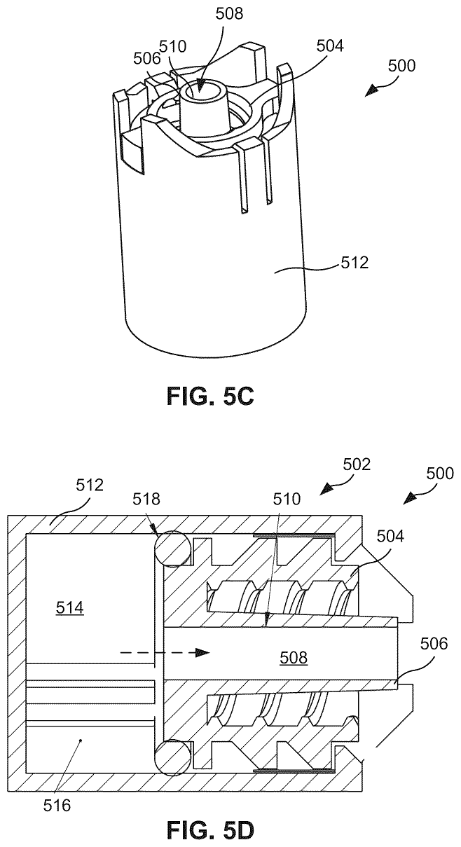

Examples catheter caps of FIG. 2A-2D, 3A-3F, and 4A-4G may receive a catheter lock solution such that an antimicrobial agent may migrate into the catheter lock solution. However, in some example catheter caps, such as described in FIG. 5A-5D, a catheter lock solution may be included in the catheter cap prior to connecting the catheter cap to a catheter luer.

FIG. 5A is a perspective view diagram of an example antimicrobial cap 500 that includes an antimicrobial agent on an insert in an uncompressed state, while FIG. 5B is a cross-sectional side view diagram of the example antimicrobial cap 500 of FIG. 5A. Cap 500 includes a housing 502. Unless otherwise indicated, housing 502 includes a connector housing 504, a hollow projection 506, an inner cavity 508, and a radially inner surface 510 similar to connector housing 204, hollow projection 206, inner cavity 208, and radially inner surface 210 of FIGS. 2A and 2B.

Housing 502 includes an outer housing 512 that defines a proximal cavity 514 at a proximal end of housing 502. Proximal cavity 514 is in fluid communication with inner cavity 508 of hollow projection 506. Connector housing 504 is configured to move relative to outer housing 512 along an axis. An O-ring 518 may be positioned against connector housing 504 along an inner surface of outer housing 512 to seal proximal cavity 514, while a limiting member 520 may limit movement of connector housing 504 within outer housing 512 at one or more positions.

Proximal cavity 514 includes a catheter lock solution and the antimicrobial agent dissolved in the catheter lock solution. Inner cavity 508 of hollow projection 506 is configured to deliver the catheter lock solution from proximal cavity 514 to the catheter luer. In the example of FIGS. 5A and 5B, cap 500 includes an insert 516 in proximal cavity 514 that includes the catheter lock solution and the antimicrobial agent. In response to the relative movement of connector housing 504 and outer housing 512, the antimicrobial agent and the catheter lock solution are configured to release from proximal cavity 514.

FIG. 5C is a perspective view diagram of an example antimicrobial cap that includes an antimicrobial agent in a proximal cavity in a compressed state, while FIG. 5D is a cross-sectional side view diagram of the example antimicrobial cap of FIG. 5C. As shown in FIGS. 5C and 5D, proximal cavity 514 is compressed relative to proximal cavity 514 of FIGS. 5A and 5B.

Insert 516 may include any of a variety of inserts configured to contain the catheter lock solution and the antimicrobial agent and release the catheter lock solution and the antimicrobial agent in response to compression. In some examples, insert 516 includes a foam positioned within proximal cavity 514, such that the antimicrobial agent is included in the foam. For example, the foam may correspond to foam 362 of FIG. 3F or foam 442 of FIG. 4E. The foam may be saturated with the catheter lock solution with dissolved antimicrobial agent and release the catheter lock solution in response to the compression. In some examples, insert 516 includes a pierceable enclosure positioned within proximal cavity 514, such that the pierceable enclosure includes the antimicrobial agent. For example, the pierceable enclosure may include a membrane or other structure that may be configured to contain the catheter lock solution with dissolved antimicrobial agent and rupture or be pierced in response to the compression.

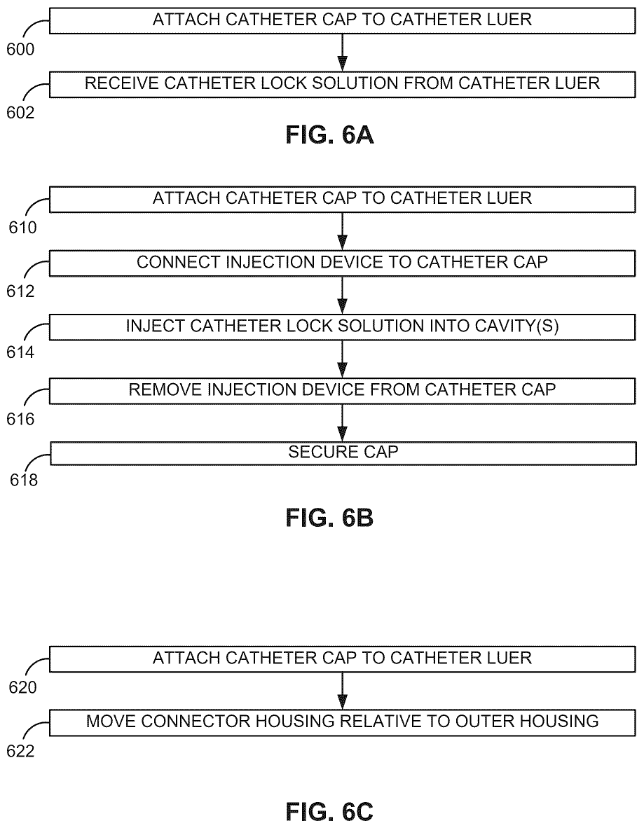

FIG. 6A is a flow diagram illustrating an example technique for disinfecting a catheter luer using an antimicrobial cap, such as caps described in FIG. 2A-D and 3A-F, that includes receiving a catheter lock solution, and will be described with respect to FIG. 2B. The method includes attaching connector housing 204 to a catheter luer that contains catheter lock solution 110 (600), thereby putting inner cavity 208 in fluid communication with the catheter luer. The method further includes receiving catheter lock solution 110 from the catheter luer. Catheter lock solution 110 contacts radially inner surface 210 and the antimicrobial agent migrates into catheter lock solution 110. Housing 202 contains catheter lock solution 110 in contact with the catheter luer to inhibit growth of microbes.

FIG. 6B is a flow diagram illustrating an example technique for disinfecting a catheter luer using an antimicrobial cap, such as caps described in FIG. 4A-4G, that includes injecting a catheter lock solution, and will be described with respect to FIG. 4B. The method includes attaching distal connector housing 404 to a catheter luer (610), thereby putting proximal cavity 414 in fluid communication with the catheter luer via inner cavity 408. The method further includes connecting an injection device to proximal connector housing 412 (612) and injecting catheter lock solution 110 into proximal cavity 414 (614). Catheter lock solution 110 contacts inner surfaces 417 of insert 416 and the antimicrobial agent migrates into catheter lock solution 110. The method further includes removing the injection device from proximal connector housing 412 (616) and attaching cap 424 (618), such that housing 402 contains catheter lock solution 110 in contact with the catheter luer to inhibit growth of microbes.

FIG. 6C is a flow diagram illustrating an example technique for disinfecting a catheter luer using an antimicrobial cap, such as caps described in FIG. 5A-D, that includes releasing a catheter lock solution, and will be described with respect to FIGS. 5B and 5D. The method includes attaching connector housing 504 to a catheter luer (620), thereby putting proximal cavity 514 in fluid communication with the catheter luer via inner cavity 508. The method further includes moving connector housing 504 relative to outer housing 512 to compress insert 516 in proximal cavity 514 (622). Catheter lock solution 110 is released from insert 516 in response to the compression. Housing 502 contains catheter lock solution 110 in contact with the catheter luer to inhibit growth of microbes.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. From the foregoing, it will be appreciated that specific examples of the present disclosure have been described herein for purposes of illustration, but that various modifications may be made without deviating from the present disclosure. For example, while particular features of the catheter caps were described as being part of a single device, in other examples, these features can be included on one or more separate devices that can be positioned adjacent to and/or used in tandem.

Certain aspects of the present disclosure described in the context of particular examples may be combined or eliminated in other embodiments. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Example 1: A catheter cap includes a housing defining at least one cavity and an opening at a distal end of the housing, wherein the housing is configured to couple to a catheter luer at the distal end, and wherein the at least one cavity is configured to contain a catheter lock solution in contact with the catheter luer at the opening; and an antimicrobial agent within the at least one cavity, wherein the antimicrobial agent is present in an amount sufficient to maintain a minimum inhibitory concentration (MIC) of the antimicrobial agent in the catheter lock solution for a period of time between successive operations of the catheter luer.

Example 2: The catheter cap of example 1, wherein the at least one cavity is configured to receive the catheter lock solution from the catheter luer, and wherein the antimicrobial agent is coated on a surface of the at least one cavity.

Example 3: The catheter cap of example 2, wherein the housing further comprises a hollow projection defining an inner cavity in fluid communication with the opening.

Example 4: The catheter cap of example 3, wherein the antimicrobial agent is coated on a radially inner surface of the hollow projection.

Example 5: The catheter cap of any of examples 3 and 4, wherein the hollow projection extends distally past the opening at the distal end of the housing.

Example 6: The catheter cap of any of examples 3 through 5, wherein the at least one cavity includes a back cavity proximal to and in fluid communication with the inner cavity of the hollow projection, and wherein the antimicrobial agent is coated on a surface of the back cavity.

Example 7: The catheter cap of any of examples 2 through 6, wherein the at least one cavity includes a proximal cavity at a proximal end of the housing and in fluid communication with the opening, and wherein the antimicrobial agent is coated on a surface within the proximal cavity.

Example 8: The catheter cap of example 7, further comprising an insert positioned within the proximal cavity, wherein the antimicrobial agent is coated on a surface of the insert.

Example 9: The catheter cap of example 8, wherein the insert includes at least one of a plurality of fins, a plurality of tubes, a film, or a foam.

Example 10: The catheter cap of any of examples 1 through 9, wherein the at least one cavity further includes a proximal cavity in fluid communication with the opening, wherein the housing is configured to interface with an injection device at a proximal end of the housing and receive the catheter lock solution from the injection device into the proximal cavity of the housing.

Example 11: The catheter cap of example 10, further comprising an insert positioned within the proximal cavity, wherein the antimicrobial agent is coated on a surface of the insert.

Example 12: The catheter cap of example 11, wherein the insert includes at least one of a plurality of fins, a film, or a foam.

Example 13: The catheter cap of any of examples 1 through 12, wherein the antimicrobial agent comprises a precipitated powder coating.

Example 14: The catheter cap of any of examples 1 through 13, wherein the antimicrobial agent is incorporated into a polymeric coating configured to release the antimicrobial agent over the period of time.

Example 15: The catheter cap of any of examples 1 through 14, wherein the at least one cavity further includes a proximal cavity at a proximal end of the housing and in fluid communication with the opening, wherein the proximal cavity includes a catheter lock solution and the antimicrobial agent dissolved in the catheter lock solution, and wherein the inner cavity of the hollow projection is configured to deliver the catheter lock solution to the catheter luer.

Example 16: The catheter cap of example 15, wherein the housing includes an outer housing and a connector housing configured to move relative to the outer housing within the proximal cavity along an axis, and wherein the antimicrobial agent and the catheter lock solution are configured to release from the proximal cavity in response to movement of the connector housing within the proximal cavity.

Example 17: The catheter cap of any of examples 15 and 16, further comprising a foam positioned within the proximal cavity, wherein the antimicrobial agent is included in the foam.

Example 18: The catheter cap of any of examples 15 through 17, further comprising a pierceable enclosure positioned within the proximal cavity, wherein the pierceable enclosure includes the antimicrobial agent.

Example 19: The catheter cap of any of examples 1 through 18, wherein a volume of the at least one cavity is less than one milliliter.

Example 20: The catheter cap of any of examples 1 through 19, wherein the antimicrobial agent is present in an amount sufficient to maintain the MIC of 16 micrograms per milliliter (μg/mL).

Example 21: The catheter cap of any of examples 1 through 20, wherein the antimicrobial agent comprises at least one of rifampin, chlorhexidine acetate, minocycline, cefazolin, gentamicin, silver glass, or silver salt.

Example 22: The catheter cap of example 21, wherein the antimicrobial agent is present in an amount sufficient to maintain the MIC of 1 μg/mL for rifampin, 20 milligrams per milliliter (mg/mL) for chlorhexidine, or 50 mg/ml for one of minocycline, cefalin, or gentamicin.

Example 23: The catheter cap of any of examples 1 through 22, wherein the period of time between the successive operations is at least one day.

Example 24: The catheter cap of example 23, wherein the period of time between the successive operations is at least three days.

Example 25: A method for fabricating a catheter cap includes positioning an antimicrobial agent in at least one cavity of a housing, wherein the housing defines an opening at a distal end of the housing and is configured to couple to a catheter luer at the distal end, wherein the at least one cavity is configured to contain a catheter lock solution in contact with the catheter luer at the opening, and wherein the antimicrobial agent is present in an amount sufficient to maintain a minimum inhibitory concentration (MIC) of the antimicrobial agent in the catheter lock solution for a period of time between successive operations of the catheter luer.

Example 26: The method of example 25, wherein positioning the antimicrobial agent comprises: injecting a solution into the at least one cavity, wherein the solution comprises the antimicrobial agent dissolved in a solvent; and removing the solvent to precipitate the antimicrobial agent onto a surface of the at least one cavity.

Example 27: A method for disinfecting a catheter luer using a catheter cap includes attaching a distal end of a housing of the catheter cap to the catheter luer, wherein the housing defines at least one cavity and an opening at a distal end of the housing, wherein the at least one cavity contains a catheter lock solution in contact with the catheter luer at the opening; wherein, prior to attaching the distal end of the housing, the at least one cavity includes an antimicrobial agent in an amount sufficient to maintain a minimum inhibitory concentration (MIC) of the antimicrobial agent in the catheter lock solution for a period of time between successive operations of the catheter luer.

Example 28: The method of example 27, wherein the at least one cavity further includes a proximal cavity at a proximal end of the housing and in fluid communication with the opening, wherein the antimicrobial agent is positioned within the proximal cavity, wherein the housing includes an outer housing and a connector housing configured to move relative to the outer housing within the proximal cavity along an axis, wherein the antimicrobial agent is configured to release in response to movement of the connector housing within the proximal cavity, and wherein the method further comprises moving the connector housing within the proximal cavity to release the antimicrobial agent.

Example 29: The method of any of examples 27 and 28, wherein the at least one cavity further includes a proximal cavity in fluid communication with the opening, wherein the antimicrobial agent is positioned within the proximal cavity, wherein the housing is configured to interface with an injection device at a proximal end of the housing and receive the catheter lock solution from the injection device into the proximal cavity of the housing, and wherein the method further comprises: attaching the injection device to the housing; and injecting the catheter lock solution into the proximal cavity using the injection device.

What is claimed is:

1. A catheter cap, comprising:
a housing defining at least one cavity and an opening at a distal end of the housing, wherein the housing is configured to couple to a catheter luer at the distal end, and wherein the at least one cavity is configured to contain a catheter lock solution in contact with the catheter luer at the opening; and
an antimicrobial agent within the at least one cavity, wherein the antimicrobial agent is present in an amount sufficient to maintain a minimum inhibitory concentration (MIC) of the antimicrobial agent in the catheter lock solution for a period of time between successive operations of the catheter luer.

2. The catheter cap of claim 1,
wherein the at least one cavity is configured to receive the catheter lock solution from the catheter luer, and
wherein the antimicrobial agent is coated on a surface of the at least one cavity.

3. The catheter cap of claim 2, wherein the housing further comprises a hollow projection defining an inner cavity in fluid communication with the opening.

4. The catheter cap of claim 3, wherein the antimicrobial agent is coated on a radially inner surface of the hollow projection.

5. The catheter cap of claim 3, wherein the hollow projection extends distally past the opening at the distal end of the housing.

6. The catheter cap of claim 3,
wherein the at least one cavity includes a back cavity proximal to and in fluid communication with the inner cavity of the hollow projection, and
wherein the antimicrobial agent is coated on a surface of the back cavity.

7. The catheter cap of claim 2,
wherein the at least one cavity includes a proximal cavity at a proximal end of the housing and in fluid communication with the opening, and
wherein the antimicrobial agent is coated on a surface within the proximal cavity.

8. The catheter cap of claim 7, further comprising an insert positioned within the proximal cavity,
wherein the antimicrobial agent is coated on a surface of the insert, and
wherein the insert includes at least one of a plurality of fins, a plurality of tubes, a film, or a foam.

9. The catheter cap of claim 1,
wherein the at least one cavity further includes a proximal cavity in fluid communication with the opening,
wherein the housing is configured to interface with an injection device at a proximal end of the housing and receive the catheter lock solution from the injection device into the proximal cavity of the housing.

10. The catheter cap of claim 1, wherein the antimicrobial agent comprises a precipitated powder coating.

11. The catheter cap of claim 1, wherein the antimicrobial agent is incorporated into a polymeric coating configured to release the antimicrobial agent over the period of time.

12. The catheter cap of claim 3,
wherein the at least one cavity further includes a proximal cavity at a proximal end of the housing and in fluid communication with the opening,
wherein the proximal cavity includes a catheter lock solution and the antimicrobial agent dissolved in the catheter lock solution, and
wherein the inner cavity of the hollow projection is configured to deliver the catheter lock solution to the catheter luer.

13. The catheter cap of claim 12,
wherein the housing includes an outer housing and a connector housing configured to move relative to the outer housing within the proximal cavity along an axis, and
wherein the antimicrobial agent and the catheter lock solution are configured to release from the proximal cavity in response to movement of the connector housing within the proximal cavity.

14. The catheter cap of claim 12, further comprising at least one of:
a foam positioned within the proximal cavity, wherein the antimicrobial agent is included in the foam; or
a pierceable enclosure positioned within the proximal cavity, wherein the pierceable enclosure includes the antimicrobial agent.

15. The catheter cap of claim 1, wherein a volume of the at least one cavity is less than one milliliter.

16. The catheter cap of claim 1, wherein the antimicrobial agent comprises at least one of rifampin, chlorhexidine acetate, minocycline, cefazolin, gentamicin, silver glass, or silver salt.

17. The catheter cap of claim 16, wherein the antimicrobial agent is present in an amount sufficient to maintain the MIC of 1 μg/mL for rifampin, 20 milligrams per milliliter (mg/mL) for chlorhexidine, or 50 mg/ml for one of minocycline, cefalin, or gentamicin.

18. The catheter cap of claim 1, wherein the period of time between the successive operations is at least one day.

19. A method for fabricating a catheter cap, comprising:
positioning an antimicrobial agent in at least one cavity of a housing,
wherein the housing defines an opening at a distal end of the housing and is configured to couple to a catheter luer at the distal end,
wherein the at least one cavity is configured to contain a catheter lock solution in contact with the catheter luer at the opening, and
wherein the antimicrobial agent is present in an amount sufficient to maintain a minimum inhibitory concentration (MIC) of the antimicrobial agent in the catheter lock solution for a period of time between successive operations of the catheter luer.

20. A method for disinfecting a catheter luer using a catheter cap, comprising:
attaching a distal end of a housing of the catheter cap to the catheter luer,
wherein the housing defines at least one cavity and an opening at a distal end of the housing,
wherein the at least one cavity contains a catheter lock solution in contact with the catheter luer at the opening;
wherein, prior to attaching the distal end of the housing, the at least one cavity includes an antimicrobial agent in an amount sufficient to maintain a minimum inhibitory concentration (MIC) of the antimicrobial agent in the catheter lock solution for a period of time between successive operations of the catheter luer.
